(19) United States
(12) Patent Application Publication
(10) Pub. No.: US 2008/0031973 A1
(43) Pub. Date: Feb. 7, 2008

(54) Means of improving hand grip

(76) Inventor: Soon Myoung Lee, Riverside, CA (US)

Correspondence Address: Soon M. Lee, 15830 Lindina Drive, Riverside, CA, 92504, US

(21) Appl. No.: 11/500,155

(22) Filed: Aug. 5, 2006

(51) Int. Cl. A01N 59/26; A61K 33/12; A61K 33/42

(52) U.S. Cl. 424/603; 424/683

(57) ABSTRACT

An antislip agent directly applicable to the hands for a short-term negation of the effects of perspiration on grip security. The hands will also become slightly sticky for 1 to 2 minutes upon use, after which time the effect of this invention will disappear, allowing one to then decide whether continued use is helpful in the setting, e.g. a strong grip was required for a batter's swings in a baseball game but is not required at other times in the game. The composition can be made in solid or liquid form.

### REFERENCES CITED

U.S. Patent Documents

Inventor: Jakob Schuler U.S. Pat. No. 4,563,218

### STATEMENT REGARDING FEDERALLY SPONSORED RESEARCH

Not Applicable

### REFERENCE TO A COMPUTER PROGRAM LISTING COMPACT DISC APPENDIX

Not Applicable

### BACKGROUND OF THE INVENTION

This invention relates to a means of temporarily enhancing the security of a hand grip on a variety of items, including but not limited to sports equipment. In particular, this invention is meant to be applied directly to the fingers and palms of one's hands, after which the amount of sweat and oil present will decrease sharply and the affected areas will become slightly sticky, allowing one to better grip an item. After 1 to 2 minutes, the effect disappears and the area of use returns to its previous state.

Many situations require the use of handheld tools whose respective applications mandate or are enhanced by a secure hand grip, and when one's hands are the primary tool, completion of a given task is near impossible without a solid grip. However, prolonged use of one's hands usually leads to an increase in perspiration in one's fingers and palms. This dilemma is a common one in sports such as tennis and golf, where sweat can severely impair the ability of a player to effectively grip the racket or club, thus reducing the accuracy of a ball strike or stroke. Several solutions to increase grip security have been proposed, the most popular of which are addressed here.

Treated gloves are often used to minimize slipperiness in construction and gardening, but are limited in their usefulness in sports because of the associated decline in tactile sensitivity and subsequent control of sports implements. Since this negatively affects accuracy and consistency, the possible improvements of which were responsible for the initial desire to have a more secure grip, gloves are impractical for use in such sports.

Treated tools, e.g. tennis rackets with handles dipped in or sprayed with chemicals to preserve grip, are currently the most prevalent means of improving an athlete's grip on a sports implement. However, because the treatment is often done by the manufacturer of the sports equipment, the level of grip enhancement is usually standardized, ignoring the differences in grip preference among players. Furthermore, the effects of the treatment can lessen quickly over time such that there exists a noticeable difference in the grip after a few weeks of moderate use. Hence, after this decline, athletes must either buy another sports implement with a newly treated grip or buy other treatments to re-enhance the grip.

Chalk is used extensively in gymnastics to minimize slipperiness of the hands but, since large amounts of chalk are needed to sufficiently absorb perspiration, application is messy and transportation is inconvenient.

An antislip agent that solves the problems presented by treated gloves, standardized equipment treatment, and chalk in reducing perspiration of the hands is presented by Jakob Schuler in U.S. Pat. No. 4,563,218. In a composition consisting of rosin, microcrystalline wax, dammar or synthetic resin, and paraffin, Schuler's invention is meant to be applied directly to the sports implement for which a more secure grip is needed. After application, he claims that it does not significantly transfer to the hand, is effective for long periods of time even in the presence of profuse perspiration, and does not damage the surface to which it is applied. Furthermore, his composition is effective as a paste, spray, or in a solid form, making it simple for an athlete to apply it repeatedly to a sports tool with the level of use appropriate for his or her aversion to slipperiness. The freedom of form also allows users to transport it easily. However, the long-term effectiveness of this antislip agent poses its own problem of decreased adaptation to variable situations.

The same sports implement may be used in a myriad of ways, some of which may necessitate a different level of grip security. Since there is a strong possibility in many sports that athletes will have to adjust their respective grips to have more or less slip in the short-run, the antislip agent proposed by Schuler may limit the variety and/or effectiveness of use that athletes have of their sports equipment. For example, a golfer striking a ball in a patch of long grass requires a much stronger grip on his or her club with less slip than the same golfer striking a ball on short grass with the same club from the same distance. If this golfer had used Schuler's antislip agent before a game in which both situations occurred, a strong possibility in the unpredictable game of golf, the effectiveness of a grip change would be limited by the composition's long-term effects. Furthermore, since the composition is impervious to perspiration and Schuler does not mention a method by which one can un-treat the sports tool, it can be inferred that it is, at best, difficult to wash off with water and, at worst, difficult to wash off at all. For the athlete who tries the antislip agent and decides against using it or applies too much and wants to undo the effects of the excess, Schuler's invention is not user-friendly.

### BRIEF SUMMARY OF THE INVENTION

The principal object of the present invention is to provide a convenient, user-friendly method of temporarily improving grip when applied to one's hands, allowing tactile sensitivity to remain unaffected. Available in both solid and liquid forms, small amounts are effective and so portability and convenience is not an issue as it has been for chalk. The improvement in grip security is greater the larger the amount applied but because the effect is temporary, lasting from 1 to 2 minutes, users can simply wait until the effect fades if they wish to lessen grip security. The composition also washes off easily with water, allowing for customization heretofore unseen in any other antislip agent. Finally, the brevity of its effects allow users to fully utilize a given tool. For example, a golfer needs a stronger grip for a long-distance shot than for a short-range putt, two shots that often occur within minutes of each other. Instead of altering the grip of his or her clubs for weeks, he or she can use this invention for the long-distance shot and have his or her hands as they were by the time the putt is required.

### BRIEF DESCRIPTION OF THE SEVERAL VIEWS OF THE DRAWING

Not Applicable

### DETAILED DESCRIPTION OF THE INVENTION

The present invention provides a method of temporarily improving grip through the direct application of said invention to one's hands. The composition is comprised of hydrous sodium lithium magnesium silicate, tetra sodium pyrophosphate, and water. Immediately after application, it has the effect of improving grip security by producing a slightly sticky feeling on the hands for 1 to 2 minutes, after which time the effect disappears and the hands return to their previous state. The effect can be adjusted

The novelty of this invention derives from the brevity of its effects, the level of customization available to the user, and convenience in application. Previous proposed solutions to the problem of a weak grip due to perspiration and natural oil in the hands aim for either a one-time application that has long-term effects on grip security or a repeatable application that has short-term effects on grip security. Those propositions of the first category raise new problems of grip adaptability among different users as well as different situations. Just as two users in the same situation may require different levels of grip security to maximize comfort, two situations presented to the same user may also require different levels of grip security. Those propositions of the second category, such as the application of chalk, have the benefit of grip adaptability but have been, in the past, messy and inconvenient to apply.

The effects of antislip compositions of this invention are fully customizable to the user in that the level of grip security achieved varies positively with the amount of the invention used. In the preferred embodiment of the invention, a small amount will produce a noticeable difference in slipperiness of the palms, but the effect can be enhanced in both strength and length with a larger dose. Furthermore, the effect can also be reduced by washing the hands with water or simply waiting 1 to 2 minutes for it to fade, as per the intention of the invention. Also, as the antislip compositions are available in solid and liquid form, users have available to them a convenient, portable formula that allows them to adapt the level of grip security to the situation within seconds of application.

The preferred embodiment of this invention utilizes hydrous sodium lithium magnesium silicate and hydrous sodium lithium magnesium silicate with tetra sodium pyrophosphate formulations obtained from Southern Clay Products, Inc. Specifically, the hydrous sodium lithium magnesium silicate used is Laponite® RD and the hydrous sodium lithium magnesium silicate with tetra sodium pyrophosphate used is Laponite® RDS, both of which are synthetic layered silicates resembling the natural clay mineral hectorite in structure and composition. These substances are nontoxic, ecologically inert, non-flammable, and approved for use in many cosmetics by international regulatory organizations.

In particular, the preferred embodiment of an antislip composition according to the invention consists of 96% by weight water, 1% by weight hydrous sodium lithium magnesium silicate in powder form from Southern Clay Products, Inc., and 3% by weight hydrous sodium lithium magnesium silicate with tetra sodium pyrophosphate in powder form from Southern Clay Products, Inc. Only the method for producing the preferred embodiment with 192 pounds of water, 2 pounds of hydrous sodium lithium magnesium silicate, and 6 pounds of hydrous sodium lithium magnesium silicate with tetra sodium pyrophosphate is discussed but it can be scaled accordingly to accommodate other amounts of the given ingredients.

To manufacture the preferred embodiment of the invention, first combine 94 pounds by weight of water with 6 pounds of RDS in powder form and 98 pounds of water with 2 pounds of RD in powder form at approximately 500 revolutions per minute in separate containers for 30 minutes and 10 minutes, respectively. Then, combine the contents of the two containers, stirring the mixture at 100 revolutions per minute for 5 minutes. At this point, the mixture will be liquid. Leave the composition in a container at room temperature for 5 days, at which point the mixture will have adopted the viscosity of a paste or cream and will remain stable at that viscosity. In other embodiments of the invention, the level of Laponite® RD may be moderated to change the viscosity of the resulting antislip composition. Furthermore, inactive ingredients may be added to change scent, color, and other superficial details not materially changing the purpose and effect of the invention.

I claim:

1. An antislip agent for direct application to the hands having the effect of increasing grip security for less than 3 minutes with one application of the recommended amount.

2. An antislip agent for direct application to the hands comprising hydrous sodium lithium magnesium silicate, hydrous sodium lithium magnesium silicate with tetra sodium pyrophosphate, and water.

3. The antislip agent defined in claim 1, comprising hydrous sodium lithium magnesium silicate, hydrous sodium lithium magnesium silicate with tetra sodium pyrophosphate, and water.

4. The antislip agent defined in claim 1, comprising 0% to 3% by weight hydrous sodium lithium magnesium silicate, 5% to 8% by weight hydrous sodium lithium magnesium silicate with tetra sodium pyrophosphate, and 93 to 98% by weight water.

5. The antislip agent defined in claim 4, which consists of 1% by weight hydrous sodium lithium magnesium silicate, 3% by weight hydrous sodium lithium magnesium silicate with tetra sodium pyrophosphate, and 94% by weight water.
